(19) United States
(12) Patent Application Publication
(10) Pub. No.: US 2026/0212773 A1
(43) Pub. Date: Jul. 23, 2026

(54) SURGICAL SKILLS TRAINER

(71) Applicant: Board of Regents, The University of Texas System, Austin, TX (US)

(72) Inventors: Daniel DeArmond, San Antonio, TX (US); John Calhoon, San Antonio, TX (US)

(21) Appl. No.: 19/452,655

(22) Filed: Jan. 19, 2026

Related U.S. Application Data
(60) Provisional application No. 63/747,063, filed on Jan. 19, 2025.

(51) Int. Cl. G09B 5/02; A61B 34/20; G09B 23/30

(52) U.S. Cl. CPC G09B 5/02; A61B 34/20; A61B 2034/2055; A61B 2034/2065; G09B 23/30

(57) ABSTRACT

A method and system for assessing surgical skills using video-tracking and efference Fast Fourier Transform (FFT) analysis of instrument movements. Sequential x,y-coordinate data from video capture is divided into geometric parcels, weighted, and merged with expected values to produce a frequency domain spectrum. Regression analysis yields R² and y-intercept values compared to ideals for real-time feedback on surgical proficiency. Applicable to laparoscopic training, tremor quantification, and incision closure, the system provides quantitative, objective assessment for skill acquisition and safety evaluation in surgical programs.

### RELATED APPLICATIONS

This Application is an International Application claiming priority to U.S. Provisional Patent Application 63/747,063 filed on Jan. 19, 2025 which is incorporated herein by reference in its entirety.

### STATEMENT REGARDING FEDERALLY FUNDED RESEARCH

None.

### FIELD

Aspects described herein are directed generally to medicine, in particular to training tools and methods.

### BACKGROUND

Maintaining quality in training and surgical outcomes is crucial. The current training equipment/methods for laparoscopic surgery need improved training and competence assessment mechanisms to ensure surgeons are adequately trained and competent in using laparoscopic equipment. The transition from traditional surgery to laparoscopic or robotic surgery requires a learning curve, which can be steep. Ensuring standardized training and assessing competence remains challenging.

The complexity of laparoscopic procedures means that even with advanced training equipment, ensuring every surgeon reaches and maintains a high standard of proficiency is difficult. Laparoscopic surgery relies on technology, which means training equipment needs to keep pace with technological advancements. This includes not just the surgical tools but also simulators and virtual reality systems for training. The rapid evolution of technology can sometimes outpace training methodologies.

While simulators are invaluable for training, replicating the exact feel and unpredictability of human tissue in a simulated environment remains a challenge. This can lead to a gap between training scenarios and real surgical conditions. The design of laparoscopic instruments and the positioning during surgery can lead to ergonomic issues for surgeons, potentially affecting long-term health. Training equipment needs to address these concerns to prevent injuries and fatigue.

Effective training requires immediate and accurate feedback. Current systems might not always provide the nuanced feedback needed for trainees to understand their mistakes or improvements needed in real-time, which is crucial for learning complex surgical techniques. Not all surgical scenarios are the same. Training equipment needs to be adaptable to various scenarios, patient anatomies, and surgical techniques, which can be a limitation in current standardized training setups.

There remains a need for additional training apparatus and methods.

### SUMMARY

Aspects described herein address the need for additional training apparatus and methods by providing a method and system for video-tracking and analysis of surgical instrument movement. The surgical instrument movement data can be analyzed using a novel Fourier transform framing algorithm to create a real-time, quantitative assessment of the success of a surgical maneuver of interest. This provides a solution to at least one or more of the following: (i) need for improved assessment of surgical skill execution that can provide real-time feedback of the totality of a surgical movement to match the time constraints and complexity of surgical conditions, (ii) need for improved assessment of the safety of surgeons-in-training for independent practice, (iii) need for improvement in endoscopic surgical skill acquisition training and assessment by surgery training programs for compliance with documentation of the curriculum milestone assessments mandated by surgical governing boards, and (iv) need for new standards in surgical skill acquisition in academic surgery programs. Novel mathematical algorithm(s) can be used to assess surgical movement in real-time and used to assess a surgeon's progress in skill acquisition. The device and system provide detailed, real-time feedback of the totality of a surgical movement which matches the time constraints and complexity of actual surgical conditions like no other current system.

Certain embodiments are directed to a method for assessing or training a surgical movement by analyzing a movement path of an object of interest comprising: (a) generating sequential x,y-coordinate pairs for an object in a video capture field during a movement exercise resulting in x,y coordinate position data; (b) dividing the movement path into geometric-area parcels, the parcels encompassing a portion of the x,y-coordinate pairs captured in video, a first geometric-area parcel covering the initial position of the movement path and the last geometric-area parcel covering the endpoint of the movement path; (c) calculating a quantitative weight for each of the geometric-area parcels based on how many x,y-coordinate pairs were captured in video in that parcel during the observed movement of the object of interest; (d) merging any 2 adjacent geometric-area parcels forming a 2× geometric-area parcel; (e) performing an efference Fast Fourier Transform (FFT) analysis by merging the observed quantitative weights for each of the geometric-area parcels against the expected values into a time domain input to produce a frequency domain of spectral bands the distribution of which can be analyzed by linear or polynomial regression to arrive at an R² and y-coordinate value; and (f) comparing the produced R² and y-coordinate value to reference values representing an ideal performance of the surgical movement and providing feedback to a user. In certain aspects at least 7, 8, 9, 10 or more parcels are designated. The geometric-area parcels can be quadrilaterals having substantially similar area. The geometric-area parcels can have the same area or contain the same length or area of a designated path of movement of an object of interest. In certain aspects the geometric-area parcels are designed so that the object of interest moving along the movement path would spend equal time in each geometric-area parcel if velocity of object of interest were constant. In certain aspects the geometric-area parcels can are designed so that the object of interest moving along the movement path would be quantified for degree of tremor by the values for the x and/or y coordinates increasing or decreasing in value when following the expected path the values for the x and/or y coordinates would have contrarily been expected to decrease or increase, respectively. In other aspects the geometric-area parcels are designed so that the object of interest consists of the edges of an open wound or some other separation of tissues undergoing repair or tissue apposition to join the edges or close the separation, where the movement path would be defined as the process of the coming-together of the separated edges and the maintenance in time of successful tissue-edge apposition. In certain embodiments the sequential x,y-coordinate pairs for an object in a video capture field and displayed on a video screen are sourced from a camera generating a video capture field from a separate video screen that comprises the video capture or virtual display of a separate camera. The video capture can be at video capture rate 10, 11, 12, 13, 14, 15, 16, 17, 18, 19, 20, 30, 50, 100 or 15, 20, 25, 30, 40, 50, 10, 150, 200 frames per second (FPS). In certain aspects the video capture is at video capture rate between 10-15 frames per second (FPS) or 100-150 frames per second (FPS), and particularly a video capture rate of 30 frames per second (FPS). In certain aspects the camera and analysis functionality are incorporated into a desktop computer, a laptop, tablet or otherwise portable computer, a smart phone, a smart watch, or smart glasses or other wearable computer device, or a robotic device including robotic or self-driving, equipment, vehicles or operating platforms.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

### Definitions

The term “object of interest” refers to any item, instrument, tissue, or feature being tracked or analyzed in the video capture field during a surgical movement exercise. This may include, but is not limited to, a surgical instrument, an instrument surrogate, a simulated tissue specimen, a manipulated object, or edges of an open wound or tissue separation.

The term “efference analysis” refers to the process of interpreting video-tracked movement data by comparing observed movement characteristics to expected or ideal movement characteristics, drawing from the neuroscience concept of efference copy. In this context, it can involve generating predicted or expected quantitative weights for geometric-area parcels and merging them with observed weights for Fast Fourier Transform (FFT) processing to assess motor skill execution, including smoothness, uniformity of velocity, and adherence to a defined path.

The term “efference Fast Fourier Transform (FFT) analysis” refers to the specific algorithmic process described herein, wherein observed quantitative weights from geometric-area parcels are interlaced with fixed expected and pad values in a time domain input (e.g., an efference window) to form a column function, followed by application of a Fast Fourier Transform to produce a frequency domain spectrum analyzable by regression (e.g., linear or polynomial) for quantitative assessment of surgical movement quality.

The term “geometric-area parcel” refers to a defined region (e.g., quadrilateral or bounding square) in the video capture field that encompasses a portion of the intended movement path, used for binning x,y-coordinate data points to calculate quantitative weights based on occupancy duration or count.

The term “quantitative weight” refers to a normalized value assigned to each geometric-area parcel, calculated as the number of x,y-coordinate pairs (or frames) occupying that parcel divided by a normalization factor (e.g., “unit”), reflecting the relative time or instances the object of interest spent in that parcel.

The term “column function” refers to the time domain signal formed by interlacing observed quantitative weights with fixed expected and pad values in the efference window, resulting in a characteristic shape resembling two adjacent impulse functions suitable for FFT analysis.

The term “R² value” refers to the coefficient of determination obtained from linear or polynomial regression analysis of the frequency domain spectrum produced by the FFT, indicating the degree of linearity (or fit) of the spectrum and serving as a measure of how closely the observed movement matches ideal uniform velocity and path adherence.

The term “y-intercept value” refers to the y-intercept obtained from regression analysis of the frequency domain spectrum, used in conjunction with the R² value to quantify movement quality against reference ideals.

The term “video capture field” refers to the area imaged by a camera and displayed on a video screen, from which sequential x,y-coordinate pairs are generated for tracking the object of interest.

The term “frames per second (FPS)” refers to the rate at which video frames are captured, influencing the temporal resolution of x,y-coordinate data generation.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of the general form of an “impulse function” if the observed movement of the surgical object of interest through the defined path has been of uniform velocity.

FIG. 2. Illustration of an impulse function with windowed (expected and pad) values, if graphed independently of the observed values.

FIG. 3A-3B. (A) Illustration of time domain data from Table 3 represented as two overlayed impulse functions. (B) Illustration of the single “column” function that results from the combining of the two impulse functions of observed and windowed values.

FIG. 4. Shows a standardized form of a graph of an FFT frequency spectrum generated from the data table shown in Table 3 and graph in FIG. 3B.

FIG. 5. Shows a list of the successive x,y coordinates for the center of an object of interest, tracked by color by video tracking software.

FIG. 6. Shows the successive x,y coordinates from FIG. 5 graphed as they would have appeared on the video screen following an arc-like path.

FIG. 7. Shows graphing of the values from Table 5 into a “column function”.

FIG. 8. Shows the FFT spectrum for the column function in FIG. 7.

FIG. 9. Shows a video screen upon completion of the movement indicating the surgeon received the message of “Signal match confirmed”.

FIG. 10. Shows successive x,y coordinates graphed from a different trial of passing the object through the arc path on a video screen.

FIG. 11. Shows the graphing of the values from Table 6 into a “column function”.

FIG. 12. Shows the FFT spectrum for the column function in FIG. 11.

FIG. 13. Shows a small video screen displaying a video capture field generated by a camera from the video source of a larger video screen from images generated by a separate video camera.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

A video camera inputs high-definition pixel data to a programmable micro-computer to track the movement of surgical instrument(s)/instrument surrogate(s). The movement is analyzed by a novel machine learning Fourier transform framing algorithm to create a real-time, mathematically based, quantitative assessment of the success of the surgical maneuver of interest. The system can be tailored by the end-user to examine a very large variety of surgical maneuvers or preferences with varying difficulty levels.

### I. Training/Assessment Methods

Skill assessment can include assessing surgical movements. Steps for movement assessment of an object in a surgical field can include tracking the movement of an object in a surgical simulation using efference analysis. One example of such movement is the movement of an object being passed in a peg transfer exercise. Efference analysis, also known as re-afference or corollary discharge analysis, is the neural process by which an organism compares an outgoing signal from the brain or nervous system terminating on muscles or other effectors for the purpose of causing movement or action, to the signal generated in a sensory organ due to the effect that movement or action has on environmental elements. Analysis in this context involves understanding or processing information based on the knowledge of what action was initiated. Efference analysis refers to the process of interpreting tracking data and assessing related motor skills (efference).

The movement of an object will generate successive x,y-coordinate pairs in a video capture field through completion of a movement; the number of pairs generated will be equal to the time to complete the movement multiplied by the frames-per-second in the video capture. The x,y-coordinate position data is divided into equal geometric-area parcels defined by x,y-coordinate bounding squares. This is a lossy step (a process or operation where some amount of the original data is intentionally discarded or altered to achieve a smaller file size or to simplify the data.); the original x,y-coordinate data is discarded and only the parcel location of a given x.y-coordinate is retained. Any number of parcels can be chosen, up to the total number of original x,y-coordinates; 7 or 8 parcels generally provide the minimum statistical power. More parcels will provide more nuance with respect to the analysis of the movement but are computationally more expensive. All parcels are weighted based on how many frames of the video the x,y-coordinates of the object remained in that parcel. Two (of the 7 or 8) parcels from a linear part of the movement geometry are merged into one parcel that contains twice the data points, i.e. twice the weight. This merging shapes the graph of the weights of the parcels into the form of a delta function or impulse function—all the parcels have a value of 1 except a single spike parcel which has a value of 2. The frequency domain graph of a delta/impulse function is a flat line which is not easily subjected to statistical analysis by a computer and is visually bland to the end user. However, interlacing two delta functions such that their spikes occur immediately adjacent in the time domain sequence, as will be performed by efference FFT windowing, results in a frequency domain spectrum which is very easy to analyze.

The interlacing of two delta functions produces a “column function” for which the broadband frequency domain graph is almost linear with a non-zero slope and thus can be analyzed by linear regression by simple visual assessment by the human end-user. If the R² value for linear regression for the curve of the frequency spectrum of the column function is above 0.9, and the y-intercept for the curve is within a certain range, it can be concluded that the movement was carried out successfully according to the programmed parameters, if not, some error in the movement was present. All of the information from the collection of x,y data points can thus be summed up in two values: the R² and the y-intercept for the curve.

If there are surgeon errors in the x,y-coordinates in the time domain they may be hard to see at the time domain level. A surgical expert or instructor may be able to see an error in movement in the time domain as it occurs but that assessment is ephemeral, subjective, and qualitative and does not necessarily provide informative feedback to help the surgeon-in-training improve. The time domain data is also not simple for a computer to analyze and apply statistical analysis to or to track improvement in surgeon skill acquisition in evolution. When subjected to what is being termed here, efference frequency analysis, success or failure in the task is easy for the end-user to assess at a glance and make adjustments to correct and easy for a computer to quantitatively score.

The efference filter provides a malleable quality-assessment tool that always programs in interlacing delta functions to normalize and standardize the visualization and statistical calculation of the quality of the movement. The efference window programs in a very small time-domain data input so the computation is fast and easy and can be done on a very small computer. End user and computer can be in accord and cross check each others' assessment of time-domain data; concrete endpoints for improvement in future practice trials can be provided; and quantitative assessment of evolution of skill acquisition over time is possible.

The steps of efference analysis of movement data are as follows:

Define a desired path of movement in a video surgical theater through which an operator would be challenged to move an object of interest—for example the arc-like movement of an object undergoing peg transfer in the Fundamentals of Laparoscopic Surgery course.

The object of interest may be a surgical instrument, a simulated tissue specimen, or a simulated tissue substrate that a surgical action will be performed on.

Identify the center of the object of interest in real-time by colorizing the object and calculating the center of the color-distribution contour of the object on the video screen using video analysis software (alternatively some other mechanism of machine learning object recognition could be substituted)

Mark the center of the object of interest by placing a computer-generated marker on the video screen which remains in the center of the object throughout its movement so the end user can always know what location the computer considers the object to be occupying at any given moment.

When defining the desired path of movement, partition the path into 8 bounding square areas that encompass the entire trajectory of the desired movement of the object of interest in the video image of the surgical theater, i.e. 8 contiguous, equal area x,y-coordinate squares that contain the full path of the desired trajectory (alternatively fewer or more partitions could be applied) where the length of the arc is equally shared between the bounding boxes.

The squares are labeled 1-8 with 1 being the first square intended to be occupied by the object of interest after initiation of the desired movement, square 8 being the last square intended to be occupied just prior to completion of the desired maneuver, and squares 4 and 5 representing the geometric center of the desired trajectory.

The squares are designed so that the object of interest moving through the defined path would spend the same amount of time in each square if the velocity of the movement were perfectly unchanging.

The design of the desired path and bounding squares does not prevent the user from moving the object in any way they wish; the definition of the intended path and bounding squares are only meant to encourage and reward efficient movement through a desired, efficient trajectory.

Prior to collecting data, squares 4 and 5 are merged to form one rectangle of twice the area of the other squares (alternatively any other two squares could be merged).

After merging there are 6 squares and 1 rectangle; squares 1-3 and 5-7 are equal in area and rectangle 4 is twice the area of the squares (alternatively fewer or more partitions could be applied).

Square 7 is now the last square intended to be occupied just prior to completion of the desired maneuver.

Since the area of rectangle 4 is twice that of the other squares, if the velocity of the movement were perfectly unchanging the object of interest moving through the defined trajectory would spend 2× the amount of time in rectangle 4 as in the other squares.

The merging of two squares represents a simple re-partitioning of the data that does not alter or exclude any data points from contributing to the calculation of the Fast Fourier Transform (FFT) but facilitates the graphical and statistical expression of efference analysis. This step can be thought of as adding a timing mechanism to the data to make it easier to characterize in terms of spectral harmonics.

The operator initiates the movement of the object of interest.

As the operator moves the object through the desired trajectory, time domain data is drawn from the sequential x,y-coordinates that the computer-calculated center of the object occupies.

Time domain data availability is limited by the video frames-per-second capture rate.

When the x,y-coordinate center of the object of interest is located within a given square/rectangle defined in step 5-11, the computer records a “range marker” specific to that square/rectangle, i.e. a “range marker” of 1 through 7.

If the center of the object is not located inside one of the 7 squares/rectangles, a “range marker” is not recorded (alternatively a “range marker” could be introduced to indicate that the object is out of bounds as a penalty).

No consideration is given to the order of “range marker” recording; any time the center of the object resides in a given square/rectangle a “range marker” specific to that square/rectangle is recorded (alternatively penalties could be added for deviation from a specific order of “range marker” recording).

No constraints are placed on the time to complete the desired trajectory, the user may take as much or as little time as they wish (alternatively time constraints could be programmed).

There are many possible variations on recording “range markers” as defined in steps 17-20 with greater or lesser stringency versus liberality in terms of judging the movement or greater or lesser nuance with respect to data analysis; the system described here is simple and allows for a liberality of movement that will still lead to successful task completion and assessment.

The “range markers” are appended by the computer to a list named “range tally”; as such, the list named “range tally” consists of a string of integers 1-7.

x,y-coordinate data is not retained except in its contribution to the “range marker” it has produced; in this way the system is “lossy” in that the exact detail of the x,y-coordinate movement is not retained for further analysis.

Movement completion is defined by the center of the object residing in a defined x,y-coordinate termination square adjacent to square 7

Upon movement completion the length of the list “range tally” is calculated

Ideally, if the movement through the squares/rectangle, 1-7, has been of unchanging velocity (smoothness of movement) the count of the integers 1-3 and 5-7 in “range tally” should be equal, and the count of the integer 4 twice that of the others; therefore, ideally, the value of the length of the list, “range tally”, divided by 8 should be equivalent to the number of each of the integers 1-3 and 5-7 expressed in “range tally” and half of the number of the integer 4 expressed in “range tally”

Divide the “range tally” list length by 8—this value is defined as unit.

The list “range tally” is broken down into 7 sub-lists which consist of the instances of the 7 integer values expressed in “range tally”, i.e., 7 separate lists for: all the instances of the integer 1 that appear in “range tally”; all the instances of the integer 2 that appear in “range tally”; and so on, continuing to all the instances of integer “7” that appear in “range tally”.

The length of all of these 7 sub-lists are calculated separately and divided separately by unit—this step normalizes the data so that the data from successive trials of movement of the object of interest can be compared.

If the movement of the object through the defined path has been of uniform velocity, the value of the length of the sub-lists divided by unit should be 1 or close to 1, except for the sub-list for the integer 4, for which the length divided by unit should have a value of 2 or close to 2; any deviation from uniform velocity will be reflected in a deviation from those ideal values of 1 and 2.

The observed time domain values are as follows:

TABLE 1

| Observed time domain | Observed time domain value |
|---|---|
| sequence position | (relative instances) |
| Observed value 1 | integer 1 instances in “range tally”/unit |
| Observed value 2 | integer 2 instances in “range tally”/unit |
| Observed value 3 | integer 3 instances in “range tally”/unit |
| Observed value 4 | integer 4 instances in “range tally”/unit |
| Observed value 5 | integer 5 instances in “range tally”/unit |
| Observed value 6 | integer 6 instances in “range tally”/unit |
| Observed value 7 | integer 7 instances in “range tally”/unit |

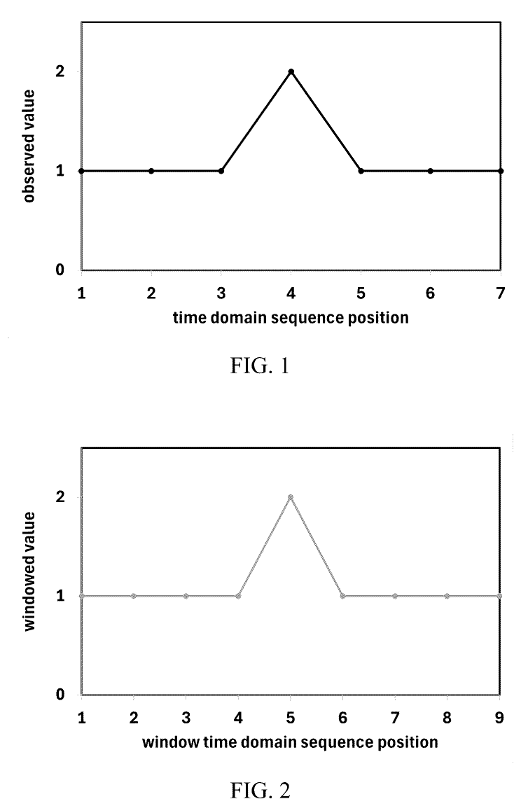

If the movement of the object through the path has been of uniform velocity and observed values 1-3 and 5-7 from Table 1 are equal to 1 or close to 1 and observed value 4 is equal to 2 or close to 2, then the graph of the values in Table 1 takes the general form of an “impulse function” shown in FIG. 1.

Movement completion as defined by the center of the object residing in a defined x,y coordinate termination is programmed to trigger the calculation and graphing of the FFT of observed versus expected values within an efference FFT window.

Insert the observed time domain values into the efference FFT window (Table 2). Observed values are variable and based on how the operator has maneuvered the object of interest through the video theater. In contrast, the windowed values are fixed and unchanging and consist of expected and pad values (bold italics cells in Table 2). Observed and windowed values are interlaced to construct the following 16-point time domain input FFT.

TABLE 2
Efference window (FFT)

| FFT | Time domain | Time domain value |
|---|---|---|
| Position | sequence position | (relative instances) |
| 1 | Pad value | 1.0 (fixed value) |
| 2 | Observed value 1 | integer 1 instances in “range tally”/unit |
| 3 | Expected value1 | 1.0 (fixed value) |
| 4 | Observed value 2 | integer 2 instances in “range tally”/unit |
| 5 | Expected value2 | 1.0 (fixed value) |
| 6 | Observed value 3 | integer 3 instances in “range tally”/unit |
| 7 | Expected value3 | 1.0 (fixed value) |
| 8 | Expected value4 | 2.0 (fixed value) |
| 9 | Observed value 4 | integer 4 instances in “range tally”/unit |
| 10 | Observed value 5 | integer 5 instances in “range tally”/unit |
| 11 | Expected value5 | 1.0 (fixed value) |
| 12 | Observed value 6 | integer 6 instances in “range tally”/unit |
| 13 | Expected value6 | 1.0 (fixed value) |
| 14 | Observed value 7 | integer 7 instances in “range tally”/unit |
| 15 | Expected value7 | 1.0 (fixed value) |
| 16 | Pad value | 1.0 (fixed value) |

If the movement of the object of interest has been adequately faithful to the defined path and of uniform velocity, the value of the length of the sub-lists divided by unit should be 1 or close to one, except for the sub-list for the integer 4, which should have a value of 2 or close to 2; in this case the populated efference FFT time domain input will be the following:

TABLE 3
Efference window (FFT)

| FFT | Time domain | Time domain value |
|---|---|---|
| Position | sequence position | (relative instances) |
| 1 | Pad value | 1.0 (fixed value) |
| 2 | Observed value 1 | 1 |
| 3 | Expected value1 | 1.0 (fixed value) |
| 4 | Observed value 2 | 1 |
| 5 | Expected value2 | 1.0 (fixed value) |
| 6 | Observed value 3 | 1 |
| 7 | Expected value3 | 1.0 (fixed value) |
| 8 | Expected value4 | 2.0 (fixed value) |
| 9 | Observed value 4 | 2 |
| 10 | Observed value 5 | 1 |
| 11 | Expected value5 | 1.0 (fixed value) |
| 12 | Observed value 6 | 1 |
| 13 | Expected value6 | 1.0 (fixed value) |
| 14 | Observed value 7 | 1 |
| 15 | Expected value7 | 1.0 (fixed value) |
| 16 | Pad value | 1.0 (fixed value) |

The windowed (expected and pad) values, if graphed independently of the observed values, will also take the form of an impulse function as shown in FIG. 2.

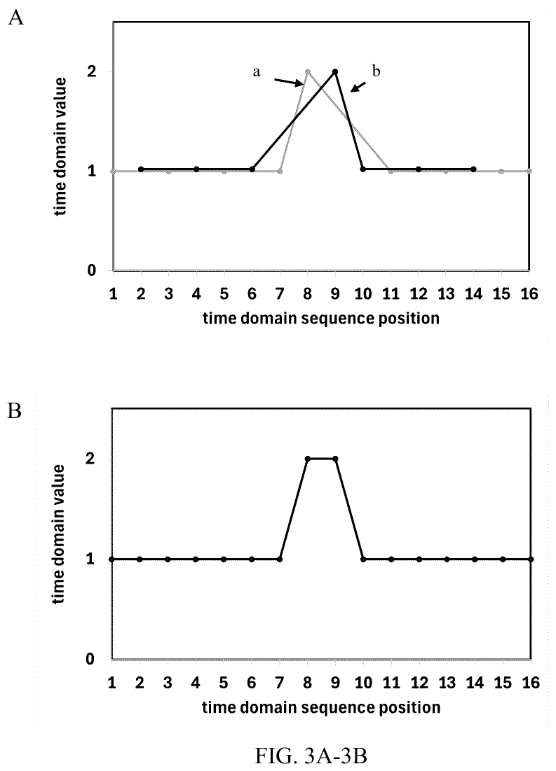

The time domain data from Table 3 may be represented as two overlayed impulse functions (FIG. 3A). This can also be expressed as an overlay of the graphs from FIG. 1 and FIG. 2. The efference window is designed so that the peak value of observed and expected impulse functions are immediately adjacent (FIG. 3A) in the time domain sequence. In FIG. 3A, the curve “a” consists of the expected/pad values which are fixed values (bold/italics cell from Table 3), and curve “b” is comprised of the observed values.

FIG. 3B shows the single “column” function that results from the combining of the two impulse functions of observed and windowed values.

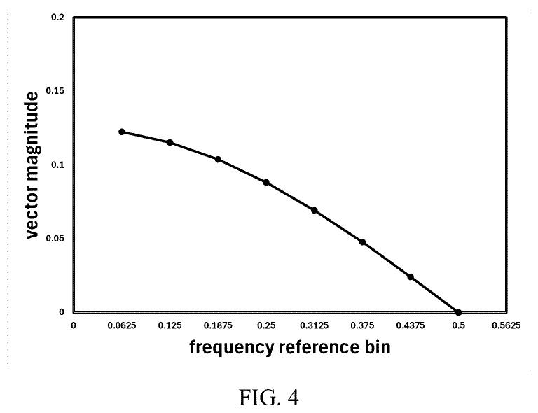

FIG. 4 shows a standardized form of a graph of an FFT frequency spectrum, as will be readily recognized by individuals knowledgeable in the art, generated from the data table shown in Table 3 and graph in FIG. 3B, where frequency is shown on the x-axis and magnitude of the vector for a given frequency is shown on the y-axis. (The vector magnitude values for frequency of 0 and frequencies greater than 0.5 are removed by convention).

FIG. 4 represents an idealized version of this curve. Observed values, being variable, will never be exactly 1 or 2 so some deviation of this perfect curve will always be observed. In its idealized form FIG. 4, the FFT of the column function, is an almost linear curve which is readily analyzable by linear regression or quadratic regression to arrive at a value for determination (R² value) of the (dependent variable) y-values versus (independent variable) x-values: for the curve in FIG. 4, the R² value for linear regression is 0.9732 and for quadratic regression is 0.994.

The use of the efference FFT allows for a holistic, integrative comparison of the observed movement of the object of interest to a defined, gold-standard path of movement. Anything less than perfect adherence to the defined path (within the allowed error) at a constant velocity throughout the entirety of the path will result in aberrations in the perfect spectral curve shown in FIG. 4. Aberrations in the curve are immediately visually appreciable to the end user and can also be quantified rapidly by the computer with either linear or quadratic regression in the form of an R² value. This allows the computer a means of independently judging the success of a movement through video tracking.

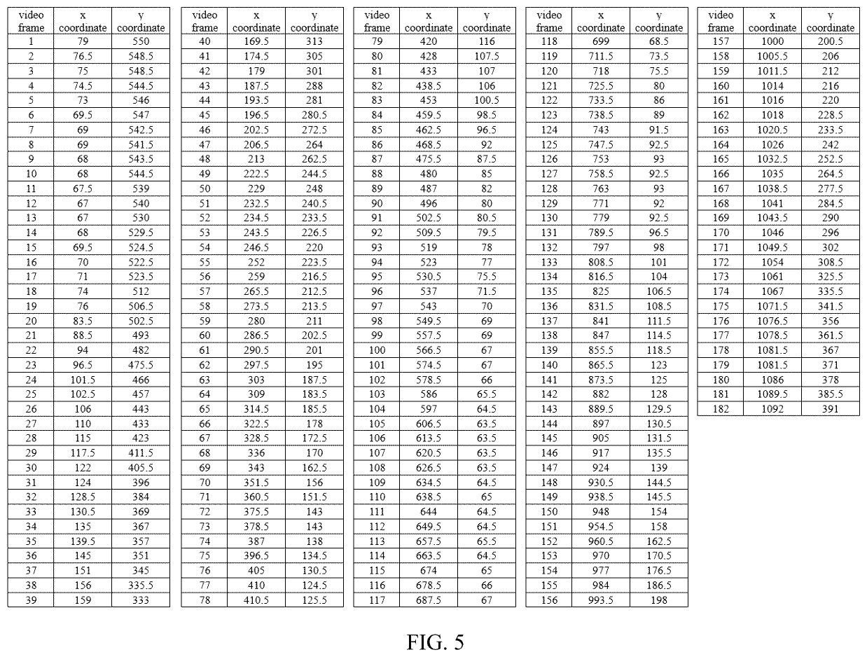

FIG. 5 shows a list of the successive x,y coordinates for the center of an object of interest, tracked by color by video tracking software. These x,y coordinate pairs were generated experimentally during the movement of the object by a laparoscopic surgical instrument through an arc path to mimic, for example, the movement of an object during a peg transfer exercise. The video screen was a 1080 by 720 pixel high-definition screen which marks x coordinates as a number between 0 and 1080 and y coordinates as a number between 0 and 720. The video capture rate was approximately 10-15FPS. A total of 182 frames were captured over a period of time of about 10-12 seconds.

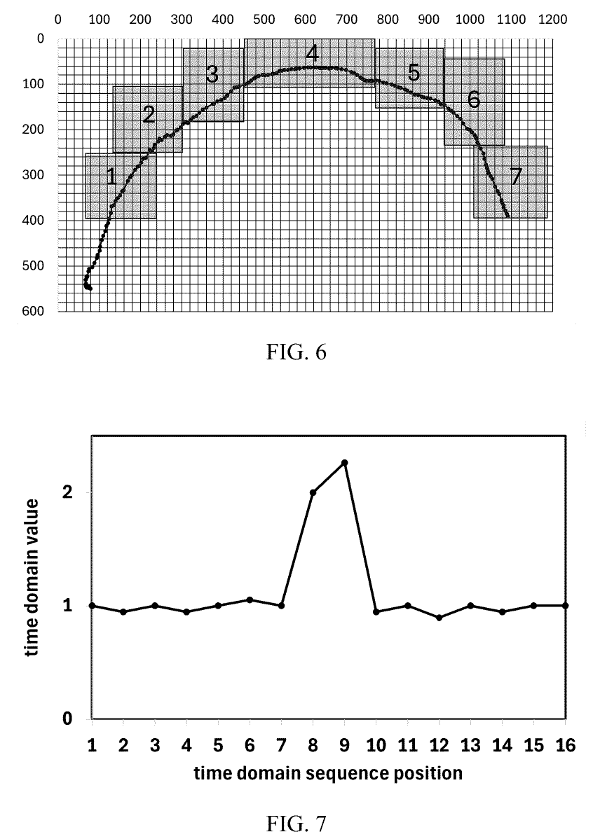

FIG. 6 shows the successive x,y coordinates from FIG. 5 graphed as they would have appeared on the video screen following an arc-like path. The values on the x and y axes are the pixel values for the video screen. The numbered squares in the figure represent the programmed parcels encompassing the path through which the object was intended to be moved. The boxes are roughly the same area and are calibrated to contain an equal segment of the arc of the intended path of movement. Note that parcel 4 contains roughly twice the length of the arc of intended movement as parcels 1-3 and 5-7.

The x,y coordinates in FIG. 5 all occupy a parcel and appear to be evenly distributed through the parcels. The range tally for this trial was calculated to be: parcel 1-18; parcel 2-18; parcel 3-20; parcel 4-43; parcel 5-18; parcel 6-17; parcel 7-18.

The value for unit was calculated as: (18+18+20+43+18+17+18)/8=19

All parcel values were divided by unit to obtain the following observed values for input into the efference FFT window.

TABLE 4

| Observed time domain | Observed time domain value |
|---|---|
| sequence position | (relative instances) |
| Observed value 1 | 0.9473 |
| Observed value 2 | 0.9473 |
| Observed value 3 | 1.0526 |
| Observed value 4 | 2.2632 |
| Observed value 5 | 0.9473 |
| Observed value 6 | 0.8947 |
| Observed value 7 | 0.9473 |

The observed values were inserted into the efference FFT window as shown in Table 5.

TABLE 5
Efference window (FFT)

| FFT | Time domain | Time domain value |
|---|---|---|
| Position | sequence position | (relative instances) |
| 1 | Pad value | 1.0 (fixed value) |
| 2 | Observed value 1 | 0.9473 |
| 3 | Expected value1 | 1.0 (fixed value) |
| 4 | Observed value 2 | 0.9473 |
| 5 | Expected value2 | 1.0 (fixed value) |
| 6 | Observed value 3 | 1.0526 |
| 7 | Expected value3 | 1.0 (fixed value) |
| 8 | Expected value4 | 2.0 (fixed value) |
| 9 | Observed value 4 | 2.2632 |
| 10 | Observed value 5 | 0.9473 |
| 11 | Expected value5 | 1.0 (fixed value) |
| 12 | Observed value 6 | 0.8947 |
| 13 | Expected value6 | 1.0 (fixed value) |
| 14 | Observed value 7 | 0.9473 |
| 15 | Expected value7 | 1.0 (fixed value) |
| 16 | Pad value | 1.0 (fixed value) |

Graphing the values from Table 5 into a “column function” produces the graph in FIG. 7.

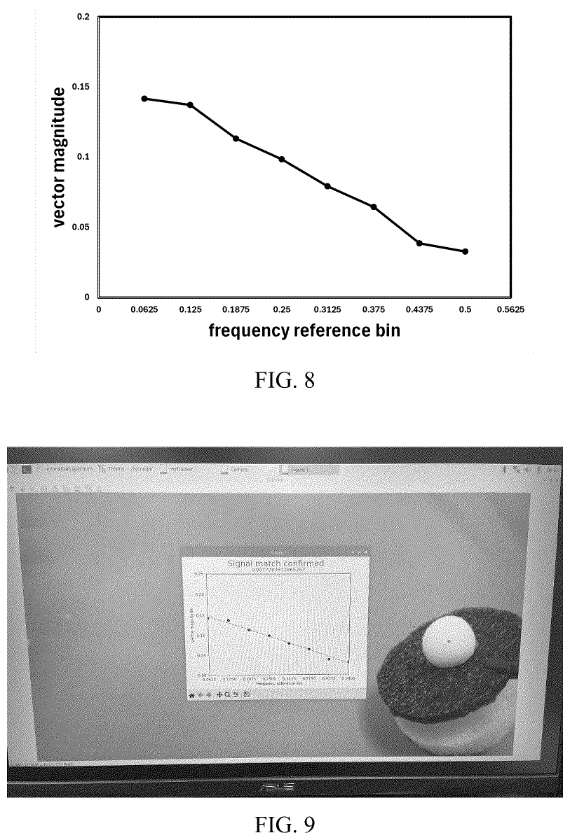

The FFT spectrum for the column function in FIG. 7 is shown in FIG. 8. The R² value for linear regression for the curve in FIG. 8 is 0.9874 and for quadratic regression is 0.9877.

The programming was designed to respond to a spectral curve with R² value greater than 0.9 with a message of successful completion of the tested surgical task. Therefore, in this case, on the video screen immediately upon completion of the movement the surgeon received the message of “Signal match confirmed” as shown in FIG. 9.

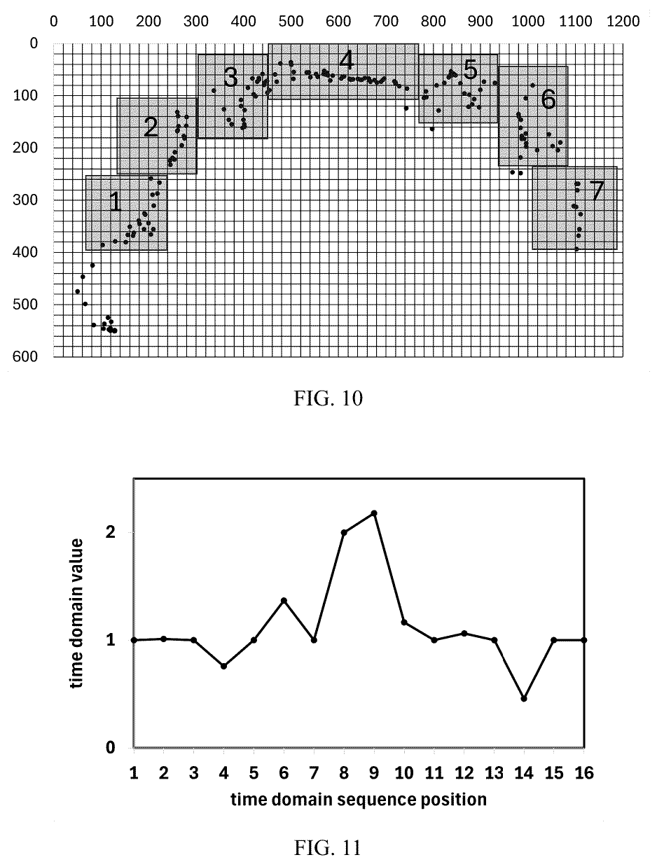

FIG. 10 shows successive x,y coordinates graphed from a different trial of passing the object through the arc path on a video screen. As in FIG. 6, the values on the x and y axes are the pixel values for the video screen and the numbered, squares in the figure represent the programmed parcels encompassing the path through which the object was intended to be moved. In this trial the x,y coordinates appear very scattered due to surgeon error or inexperience.

The range tally for this trial was calculated to be: parcel 1-20; parcel 2-15; parcel 3-27; parcel 4-43; parcel 5-23; parcel 6-21; parcel 7-9. The unit value was 19.75. The observed values for insertion into the efference FFT were: obs value 1-1.0126; obs value 2-0.7594; obs value 3-1.3671; obs value 4-2.1772; obs value 5-1.1646; obs value 6-1.0633; obs value 7-0.4557. The efference FFT table was as follows:

TABLE 6
Efference window (FFT)

| FFT | Time domain | Time domain value |
|---|---|---|
| Position | sequence position | (relative instances) |
| 1 | Pad value | 1.0 (fixed value) |
| 2 | Observed value 1 | 1.0126 |
| 3 | Expected value1 | 1.0 (fixed value) |
| 4 | Observed value 2 | 0.7594 |
| 5 | Expected value2 | 1.0 (fixed value) |
| 6 | Observed value 3 | 1.3671 |
| 7 | Expected value3 | 1.0 (fixed value) |
| 8 | Expected value4 | 2.0 (fixed value) |
| 9 | Observed value 4 | 2.1772 |
| 10 | Observed value 5 | 1.1646 |
| 11 | Expected value5 | 1.0 (fixed value) |
| 12 | Observed value 6 | 1.0633 |
| 13 | Expected value6 | 1.0 (fixed value) |
| 14 | Observed value 7 | 0.4557 |
| 15 | Expected value7 | 1.0 (fixed value) |
| 16 | Pad value | 1.0 (fixed value) |

Graphing the values from Table 6 into a “column function” produces the graph in FIG. 11.

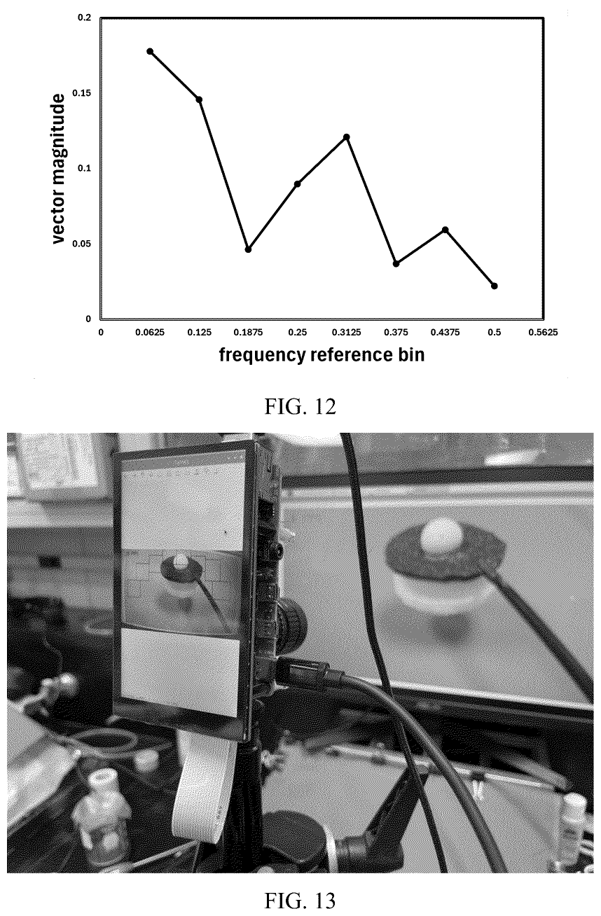

The FFT spectrum for the column function in FIG. 11 is shown in FIG. 12. The R² value for linear regression for the curve in FIG. 12 is 0.6231 and for quadratic regression is 0.6478.

The programming was designed to respond to a spectral curve with R² value greater than 0.9 with a message of successful completion of the tested surgical task. Therefore, in this case, on the video screen immediately upon completion of the movement the surgeon received the message of “Absent signal match” in a format similar to that in FIG. 9.

This shows the ability of this system to differentiate between a surgical maneuver carried out with skilled execution versus surgical error.

Instead of being trained on a surgical field and object of interest in real space, FIG. 13 shows that the camera serving as the video source for data analysis can be trained on a video source from a second video screen generated by a second video camera. FIG. 13 is a snapshot image of a camera generating a video feed and performing data analysis as described herein on a miniature computer monitor, “a”, trained on a video screen, “b”, displaying video from a second camera source. This shows that the same data analysis described above can be performed on the moving image of an object of interest depicted on a second video screen. This is an important feature in that no current surgical video sources have the data analysis capability of the system described herein. Because the system described herein can be made small and portable, it can be applied to other surgical video sources without the need to reconfigure that native video source, which may be prohibitive from cost or engineering standpoints.

In other embodiments, the efference analysis approach can be programmed to assign a score for degree of tremor in surgical motions as a statistical means of assigning a score to the amount of tremor in a surgeon's instrument handling during performance of a surgical action.

In other embodiments, the efference analysis approach can be programmed to assign a score for successful closure of an incision or tissue apposition after suture-closure to provide a statistical means of verifying incision closure or tissue apposition after suture-closure by a surgeon or autonomous surgical robot.

In other embodiments, instead of video data the efference analysis approach may be applied to sensors such as: flex sensors, position sensors, accelerometers, strain gauges, sensors requiring an analog to digital conversion, electrical sensors to provide a statistical means of verifying a defined contact, tactile, or proprioceptive sensory input by a surgeon or autonomous surgical robot. Novel mathematical algorithm to assess surgical movement in real-time that can be used to assess surgeon progress in surgical skill acquisition. This device and system provide detailed, real-time feedback of the totality of a surgical movement which matches the time constraints and complexity of actual surgical conditions like no other current system is capable of.

### II. Training System

Tracking environment includes cameras coupled to a controller/analyzer that receives instrument position data, and video capture data; green screen and the like. Training instrumentation is coupled to a sensor system, assesses three-dimensional positioning of the training instrumentation. Simulated target/situation can include characteristics of the trainer including, but is not limited to (i) uniform movement tracker, (ii) Jitter or tremor tracker, (iii) tracks evolution, (iv) tracks based on position not velocity or acceleration, (v) can track instrument, tissue, or manipulated object, and/or (vi) color recognition versus machine learning.

1. A method for assessing or training a surgical movement by analyzing a movement path of an object of interest comprising:
(a) generating sequential x,y-coordinate pairs for an object in a video capture field during a movement exercise resulting in x,y coordinate position data;
(b) dividing the movement path into geometric-area parcels, the parcels encompassing a portion of the x,y-coordinate pairs captured in video, a first geometric-area parcel covering the initial position of the movement path and the last geometric-area parcel covering the endpoint of the movement path;
(c) calculating a quantitative weight for each of the geometric-area parcels based on how many x,y-coordinate pairs were captured in video in that parcel during the observed movement of the object of interest;
(d) merging any 2 adjacent geometric-area parcels forming a 2× geometric-area parcel;
(e) performing an efference Fast Fourier Transform (FFT) analysis by merging the observed quantitative weights for each of the geometric-area parcels against the expected values into a time domain input to produce a frequency domain of spectral bands the distribution of which can be analyzed by linear or polynomial regression to arrive at an R² and y-coordinate value; and
(f) comparing the produced R² and y-coordinate value to reference values representing an ideal performance of the surgical movement and providing feedback to a user.

2. The method of claim 1, wherein at least 7 parcels are designated.

3. The method of claim 1, wherein 8 or more parcels are designated.

4. The method of claim 1, wherein the geometric-area parcels are quadrilaterals having substantially similar area.

5. The method of claim 1, wherein the geometric-area parcels have the same area or contain the same length or area of a designated path of movement of an object of interest.

6. The method of claim 1, wherein the geometric-area parcels are designed so that the object of interest moving along the movement path would spend equal time in each geometric-area parcel if velocity of object of interest were constant.

7. The method of claim 1, wherein the geometric-area parcels are designed so that the object of interest moving along the movement path is quantified for degree of tremor by the values for the x and/or y coordinates increasing or decreasing in value when following an expected path the values for the x and/or y coordinates would have contrarily been expected to decrease or increase, respectively

8. The method of claim 1, wherein the geometric-area parcels are designed so that the object of interest consists of the edges of an open wound or some other separation of tissues undergoing repair or tissue apposition to join the edges or close the separation, where the movement path would be defined as the process of the coming-together of the separated edges and the maintenance in time of successful tissue-edge apposition

9. The method of claim 1, wherein the sequential x,y-coordinate pairs for an object in a video capture field and displayed on a video screen are sourced from a camera generating a video capture field from a separate video screen that comprises the video capture or virtual display of a separate camera

10. The method of claim 1, wherein the video capture is at video capture rate 10-15 frames per second (FPS).

11. The method of claim 1, wherein the video capture is at video capture rate 30 frames per second (FPS).

12. The method of claim 1, wherein the video capture is at video capture rate 100-150 frames per second (FPS).

13. The method of claim 1, wherein the camera and analysis functionality are incorporated into a desktop computer, a laptop, tablet or otherwise portable computer, a smart phone, a smart watch, or smart glasses or other wearable computer device, or a robotic device including robotic or self-driving, equipment, vehicles or operating platforms.
